(19) United States
(12) Patent Application Publication
(10) Pub. No.: US 2014/0309476 A1
(43) Pub. Date: Oct. 16, 2014

(54) CT ATLAS OF MUSCULOSKELETAL ANATOMY TO GUIDE TREATMENT OF SARCOMA

(71) Applicants: H. Lee Moffitt Cancer Center and Research Institute, Inc., Tampa, FL (US); University of South Florida, Tampa, FL (US)

(72) Inventors: Steven Eric Finkelstein, Scottsdale, AZ (US); Mark S. Russell, Sherman, TX (US)

(21) Appl. No.: 14/315,081

(22) Filed: Jun. 25, 2014

Related U.S. Application Data
(63) Continuation-in-part of application No. 13/336,107, filed on Dec. 23, 2011.
Application No. 13/336,107 is a continuation of application No. PCT/US2010/040168, filed on Jun. 28, 2010.
(60) Provisional application No. 61/220,729, filed on Jun. 26, 2009.

(51) Int. Cl. A61N 5/10

(52) U.S. Cl. CPC A61N 5/10
USPC 600/1

(57) ABSTRACT

The method includes the steps of obtaining atlas data in an atlas coordinate set from a computer-readable atlas of musculoskeletal structure. The atlas contains a data set representing a 3D model of musculoskeletal structure divided into the relevant musculoskeletal anatomic terminology. The method further includes obtaining patient data. The patient data is in a patient coordinate set that corresponds to obtained atlas data in the atlas coordinate set. The atlas data is then morphed by performing a rigid registration between the at least one landmark identified in the patient coordinate set and the corresponding obtained atlas data in the atlas coordinate set and by performing a non-rigid registration of the musculoskeletal structure divided into the relevant contoured fascial compartments, skeletal structures and vascular structures while maintaining the rigid registration of the at least one landmark. The image of the patient coordinate set with the morphed atlas data is then displayed.

### CROSS REFERENCE TO RELATED APPLICATIONS

This application is a continuation-in-part of currently pending U.S. patent application Ser. No. 13/336,107 filed Dec. 23, 2011, which is a continuation of prior filed International Application, Serial No. PCT/US2010/040168, filed Jun. 28, 2010, which claims priority to U.S. Provisional Patent Application No. 61/220,729, filed Jun. 26, 2009, all of which are hereby incorporated by reference into this disclosure. This application also relates to prior filed International Application, Serial No. PCT/US2010/027470, filed Mar. 16, 2010, which claims priority to U.S. Provisional Application No. 61/160,396, filed Mar. 16, 2009, all of which are hereby incorporated by reference into this disclosure.

### BACKGROUND OF THE INVENTION

The terminology of compartmental musculoskeletal anatomy is the foundation for communication among orthopedic oncologists. With the increasing use of both intensity modulated radiotherapy (IMRT) and image guided radiation techniques (IGRT) by radiation oncologists, there is an increasing need for useful radiologic correlation with surgical anatomy.

### SUMMARY OF INVENTION

The invention includes, in a general embodiment, a method for the segmentation and alignment of compartmental musculoskeletal anatomy using a computer readable atlas of compartmental musculoskeletal structure having a data set representing a three-dimensional (3D) model of compartmental musculoskeletal structure divided into the relevant musculoskeletal anatomy terminology. The atlas image, or a portion thereof defined by a coordinate set, is compared to patient data relating to musculoskeletal anatomy (or a portion thereof defined by a patient coordinate set), such as from a CT scan, that corresponds to the obtained atlas data (or vice versa). The atlas data can then be used to display the subject by overlaying and/or deforming the atlas data onto the patient data.

The invention also includes obtaining patient data in a patient coordinate set that correspond to atlas data in an atlas coordinate set by collecting a plurality of reference points in a patient coordinate set from the patient that correspond to points in an atlas coordinate set from the atlas. Alternately, the obtained patient data comprises a plurality of points from the patient anatomy in a patient coordinate set, and the obtained atlas data comprises a plurality of points from the atlas in an atlas coordinate set.

Optionally, the step of obtaining a plurality of points in a patient coordinate set that correspond to points in an atlas coordinate set from the atlas comprises (1) obtaining an image of the patient including a plurality of points in an image coordinate set that correspond to points in an atlas coordinate set from the atlas, (2) collecting a plurality of points in a patient coordinate set from the patient that correspond to points in an atlas coordinate set from the atlas (3) collecting a plurality of points in a patient coordinate set from the patient that correspond to points in an image coordinate set from the image (4) identifying at least one landmark within the plurality of points in the patient coordinate set, and (5) morphing atlas data from the atlas coordinate set to the patient coordinate set is accomplished by performing a rigid registration between the at least one landmark identified in the patient coordinate set and the corresponding obtained atlas data in the atlas coordinate set while maintaining the rigid registration of the at least one landmark.

### BRIEF DESCRIPTION OF THE DRAWINGS

For a fuller understanding of the invention, reference should be made to the following detailed description, taken in connection with the accompanying drawings, in which:

FIG. 1A is a computer-generated, skeletal, left view image of the upper extremity.

FIG. 1B is a computer-generated, skeletal, anterior view image of the upper extremity.

FIG. 2 is an anterior view of a three-dimensional musculoskeletal model of the upper extremity, according to an embodiment of the present invention.

FIG. 3 is a left view of a three-dimensional musculoskeletal model of the upper extremity, according to an embodiment of the present invention.

FIG. 4A is an inferior view of a slice (slice 20) of the upper extremity, according to an embodiment of the present invention.

FIG. 4B is a left view of a slice (slice 159) of the upper extremity, according to an embodiment of the present invention.

FIG. 4C is an anterior view of a slice (slice 200) of the upper extremity, according to an embodiment of the present invention.

FIG. 5A is an inferior view of a slice (slice 22) of the upper extremity, according to an embodiment of the present invention.

FIG. 5B is a left view of a slice (slice 159) of the upper extremity, according to an embodiment of the present invention.

FIG. 5C is an anterior view of a slice (slice 200) of the upper extremity, according to an embodiment of the present invention.

FIG. 6A is an inferior view of a slice (slice 42) of the upper extremity, according to an embodiment of the present invention.

FIG. 6B is a left view of a slice (slice 159) of the upper extremity, according to an embodiment of the present invention.

FIG. 6C is an anterior view of a slice (slice 200) of the upper extremity, according to an embodiment of the present invention.

FIG. 7A is an inferior view of a slice (slice 107) of the upper extremity, according to an embodiment of the present invention.

FIG. 7B is a left view of a slice (slice 161) of the upper extremity, according to an embodiment of the present invention.

FIG. 7C is an anterior view of a slice (slice 200) of the upper extremity, according to an embodiment of the present invention.

FIG. 8A is an inferior view of a slice (slice 139) of the upper extremity, according to an embodiment of the present invention.

FIG. 8B is a left view of a slice (slice 161) of the upper extremity, according to an embodiment of the present invention.

FIG. 8C is an anterior view of a slice (slice 200) of the upper extremity, according to an embodiment of the present invention.

FIG. 9 is an anterior view of a three-dimensional musculoskeletal model of the lower extremities, according to an embodiment of the present invention.

FIG. 10 is a right view of a three-dimensional musculoskeletal model of the lower extremity, according to an embodiment of the present invention.

FIG. 11 is a right view of a slice (slice 170) of the lower extremities, according to an embodiment of the present invention.

FIG. 12 is a superior view of a slice (slice 126) of the lower extremities, according to an embodiment of the present invention.

FIG. 13 is a superior view of a slice (slice 86 of the lower extremities, according to an embodiment of the present invention.

FIG. 14 is a superior view of a slice (slice 34) of the lower extremities, according to an embodiment of the present invention.

FIG. 15 is a superior view of a slice (slice 4) of the lower extremities, according to an embodiment of the present invention.

### DETAILED DESCRIPTION OF THE PREFERRED EMBODIMENT

With the increasing use of both intensity-modulated radiotherapy (IMRT) and image-guided radiation techniques (IGRT) by radiation oncologists, there is an increasing need for useful radiologic correlation with surgical anatomy. IMRT enables the delivery of complex radiation therapy (RT) plans that previously could not be accomplished with conventionally planned two- to four-field techniques or more sophisticated three-dimensional (3D) conformal RT (3D-CRT). The advent of IMRT provides an opportunity to spare critical normal tissue. For patients receiving radiation for sarcoma, normal tissue may be better protected with IMRT than other conformal techniques as demonstrated by dosimetric investigations. Use of IMRT has also been associated with reduced acute toxicity for sarcoma. Critical to the use of IMRT, is a clear understanding of elective CTV targets. Treatment with IMRT demands much more detailed knowledge of target structures than the conventionally planned techniques. Indeed, WIRT utilizes customized treatment planning based on an individual's anatomy.

Compartmental musculoskeletal anatomy has now been successfully adapted into a 3D atlas for imaging and radiation treatment planning This model creates a common system that can facilitate contouring, treatment planning, and follow-up response evaluation.

Terminology that is anatomically and surgically correct, consistent, and concise was applied to comprehensively contouring structures on axial CT slices from normal and abnormal upper extremity. Software was used to reconstruct these images into a 3D data set. Anatomic accuracy was then confirmed by a multidisciplinary panel with representatives from radiology, orthopedic oncology, and radiation oncology.

Musculoskeletal models were produced by first considering the six segments of the upper extremity: the shoulder, arm, elbow, forearm, wrist, and hand. Fascial compartments were then contoured and constructed. Next, skeletal and vascular structures were added. This provided a 3D model integrating the anatomic nomenclature facilitating reliable communication between the radiologist, surgeon, and radiation oncologist.

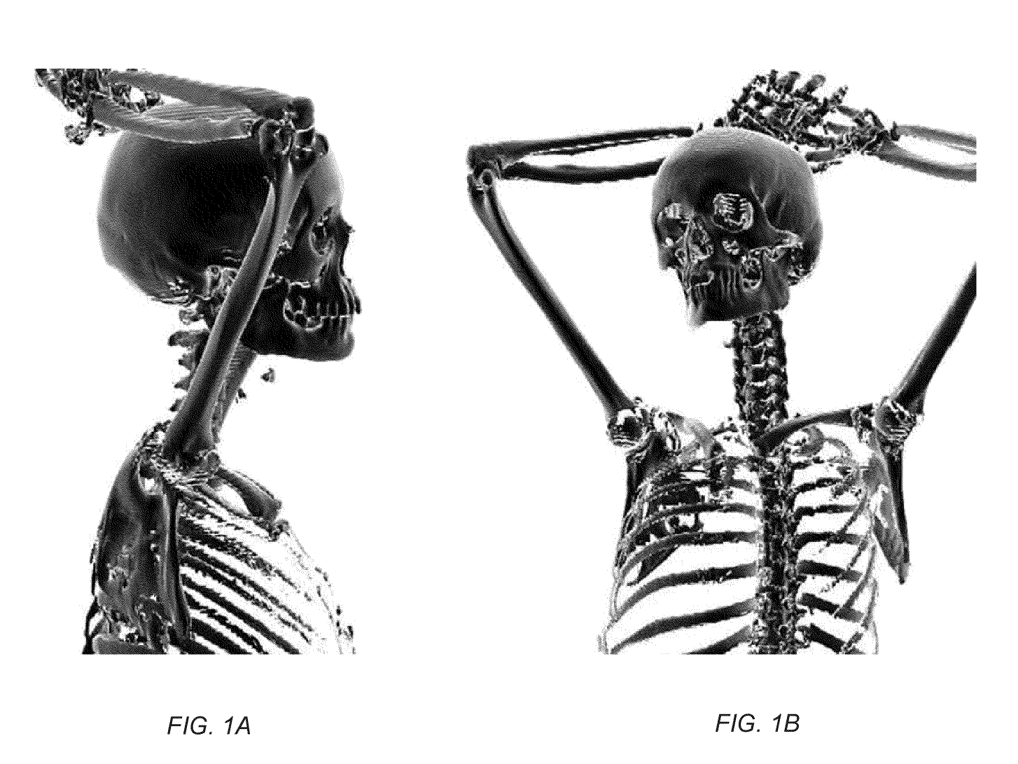

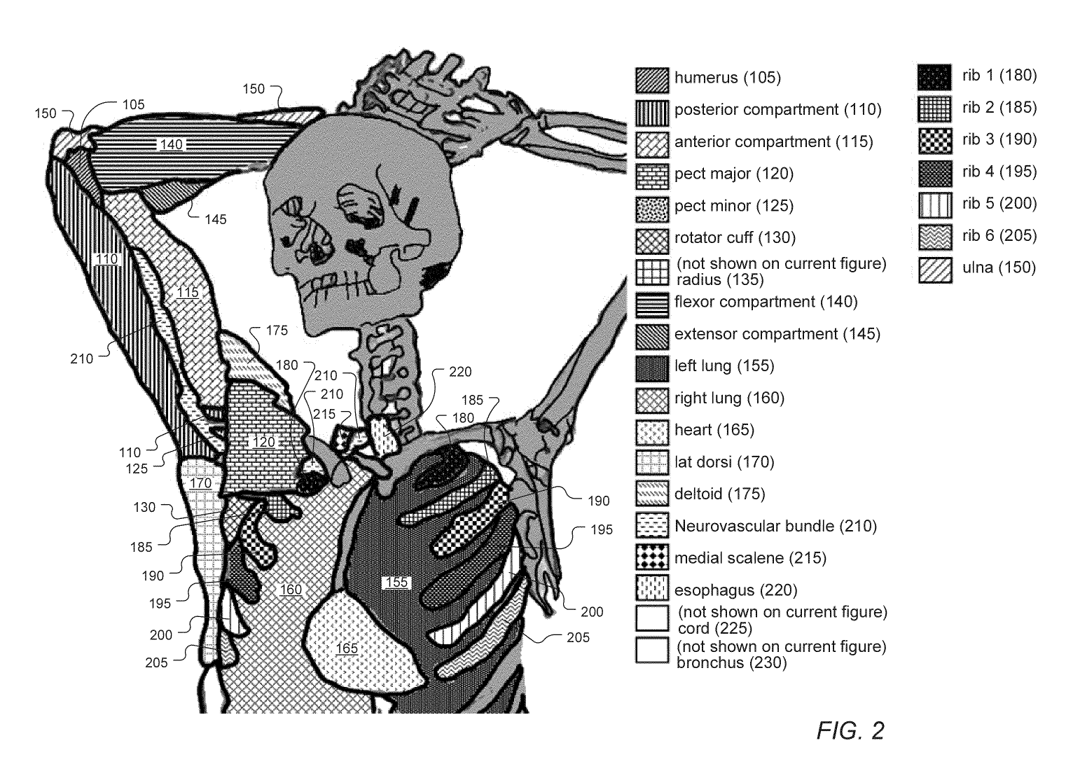

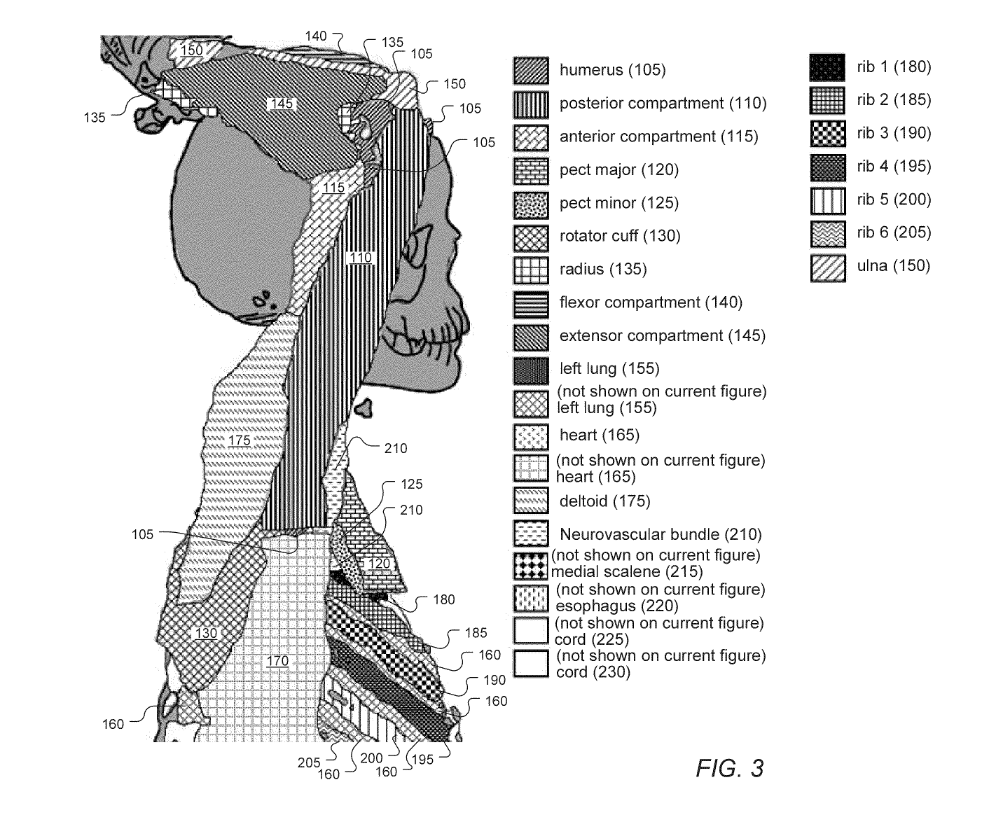

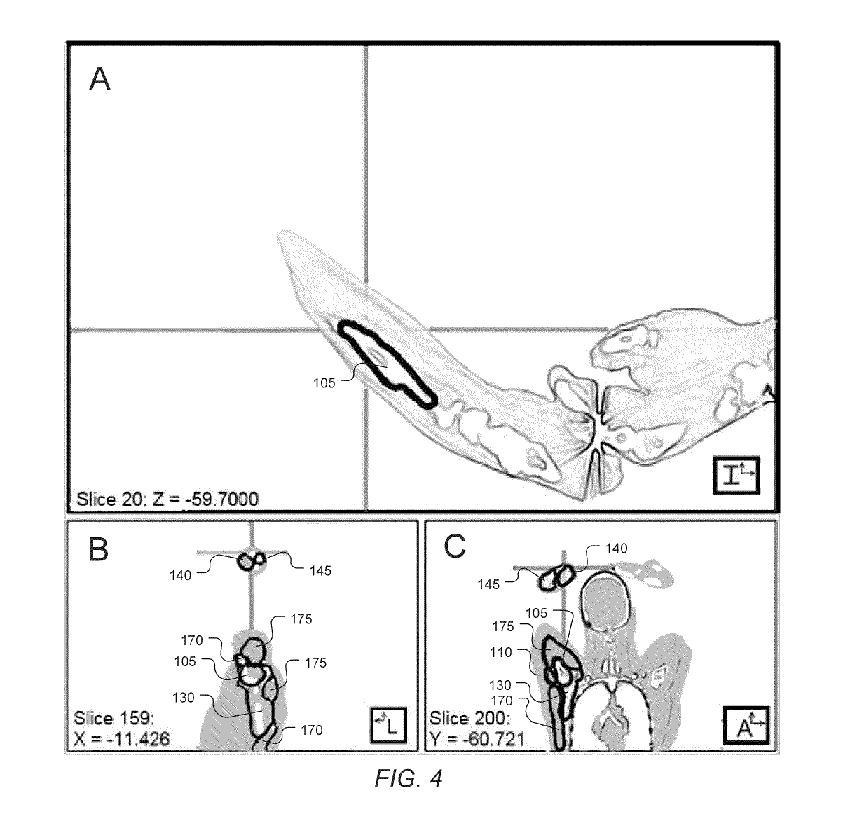

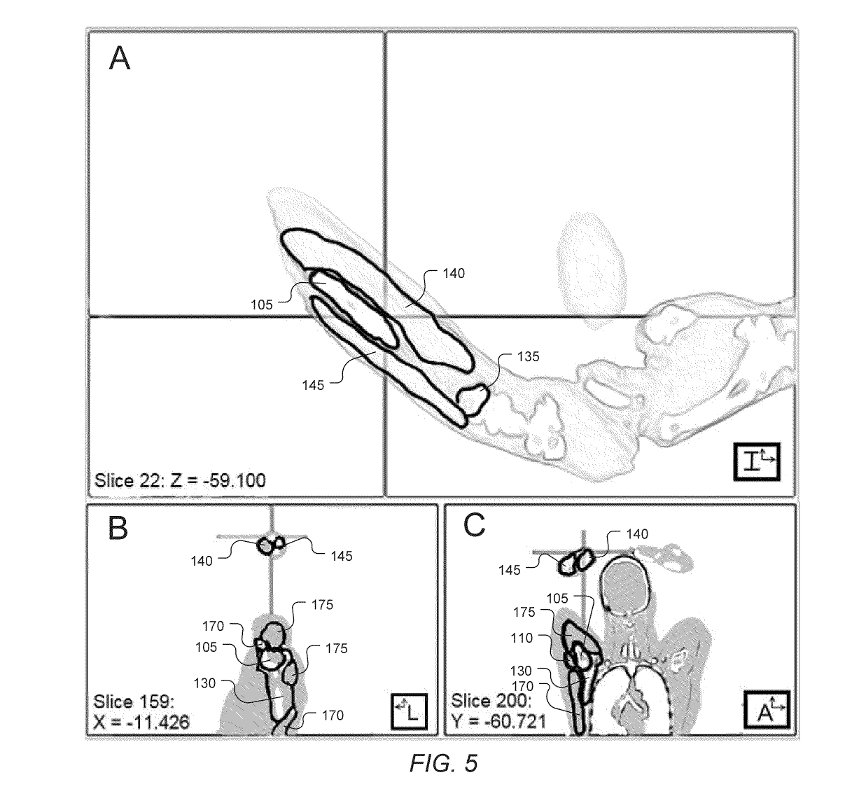

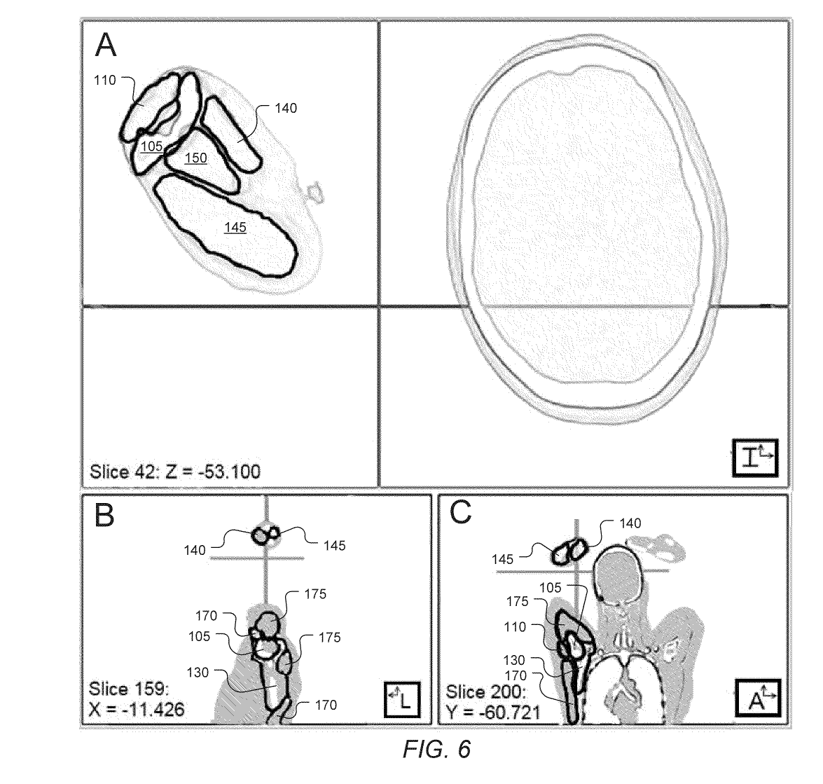

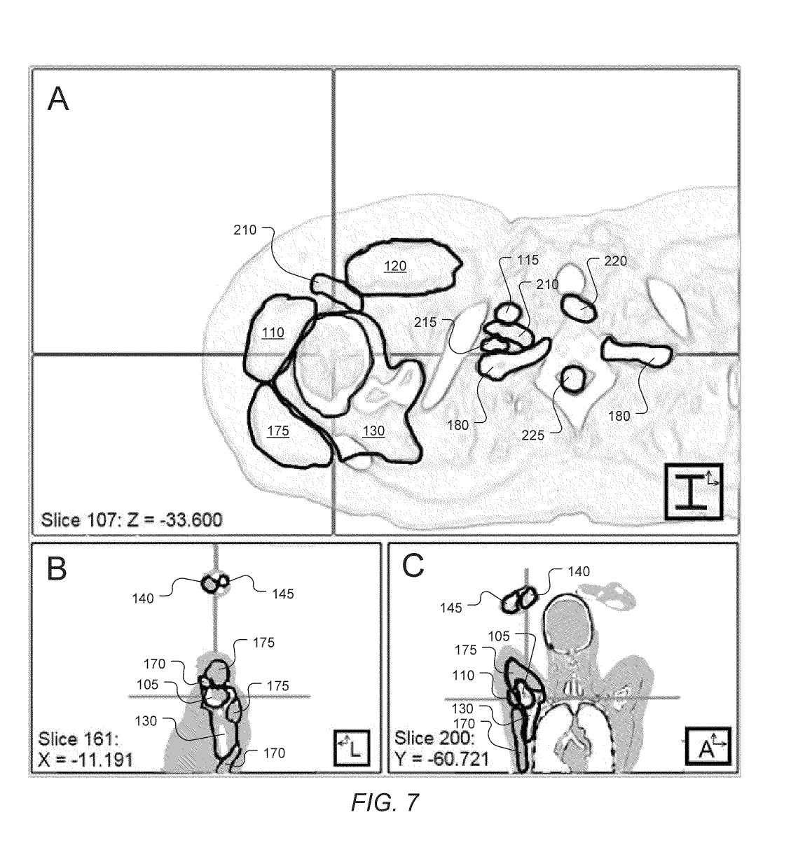

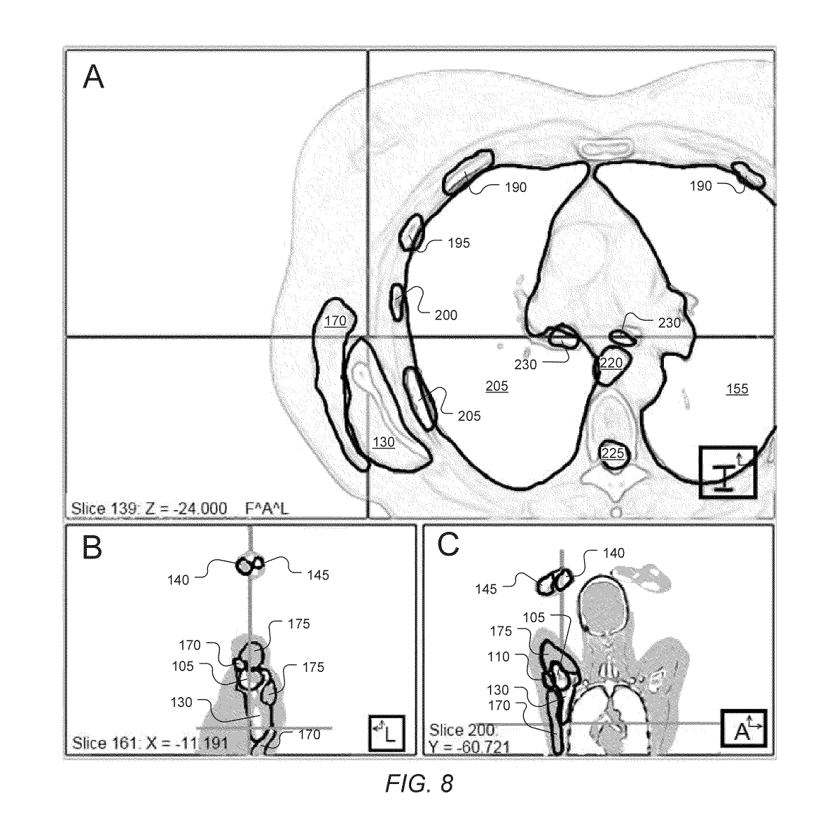

Example images of the three-dimensional (3D) CT atlas of the upper extremity for sarcoma are shown in FIGS. 1 through 8. FIGS. 1A and 1B are computer-generated, skeletal images of the upper extremity. FIG. 1A is a left view and FIG. 1B is an anterior view. FIGS. 2 and 3 are an anterior view and a left view, respectively, of a 3D musculoskeletal model of the upper extremity. FIGS. 4 through 8 are CT slices of the upper extremity emphasizing the contours of the relevant musculoskeletal structures.

Similar to the 3D CT Atlas for the upper extremity, terminology that is anatomically and surgically correct, consistent, and concise was applied to comprehensively contouring structures on axial CT slices from normal and abnormal lower extremity. Software was used to reconstruct these images into a 3D data set. Anatomic accuracy was then confirmed by a multidisciplinary panel with representatives from radiology, orthopedic oncology, and radiation oncology.

Musculoskeletal models were produced by first considering the two segments of the lower extremity: the thigh and the leg. Fascial compartments were then contoured and constructed. Next, skeletal and vascular structures were added. This provided a 3D model integrating the anatomic nomenclature facilitating reliable communication between the radiologist, surgeon, and radiation oncologist.

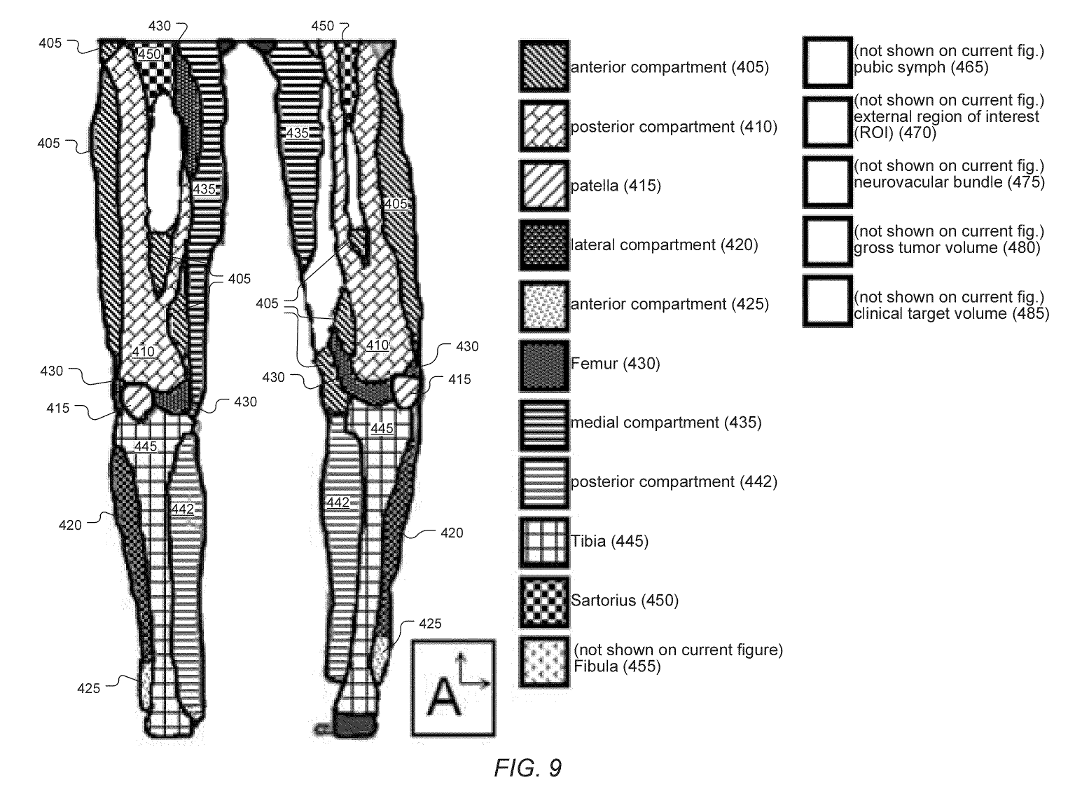

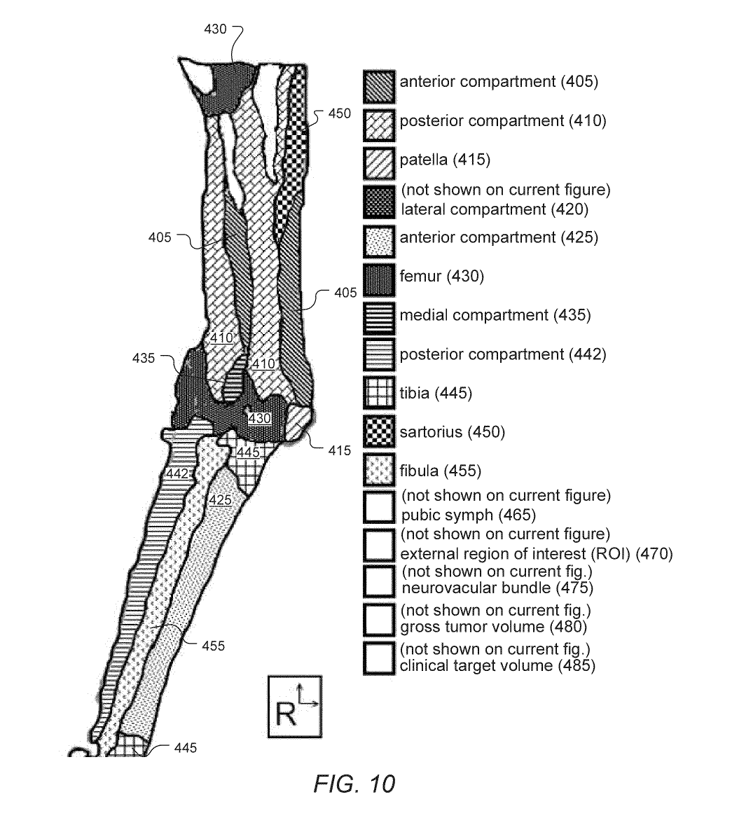

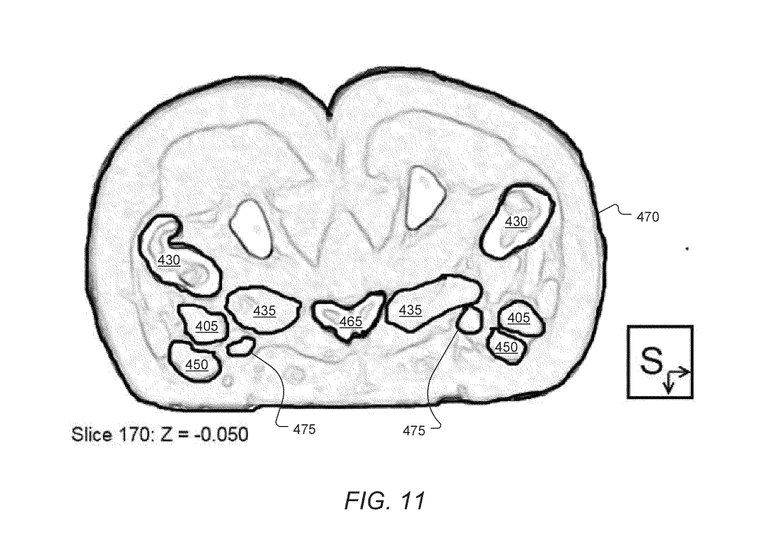

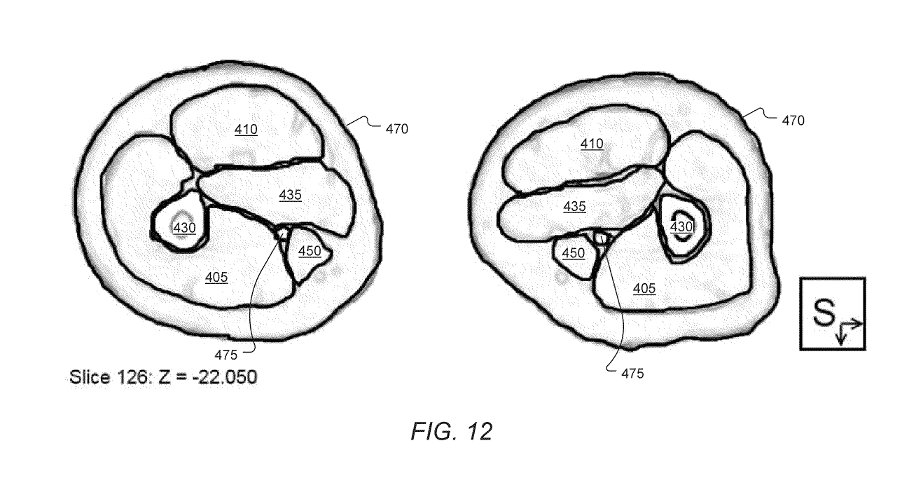

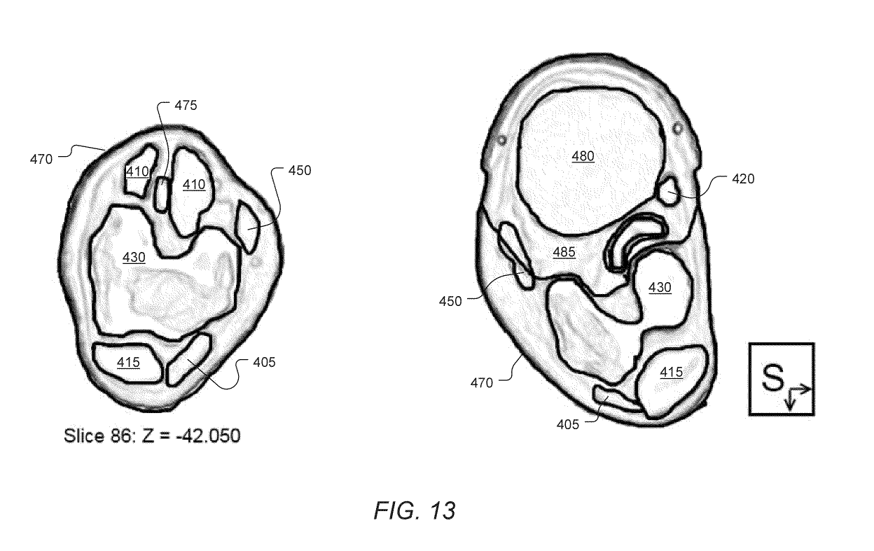

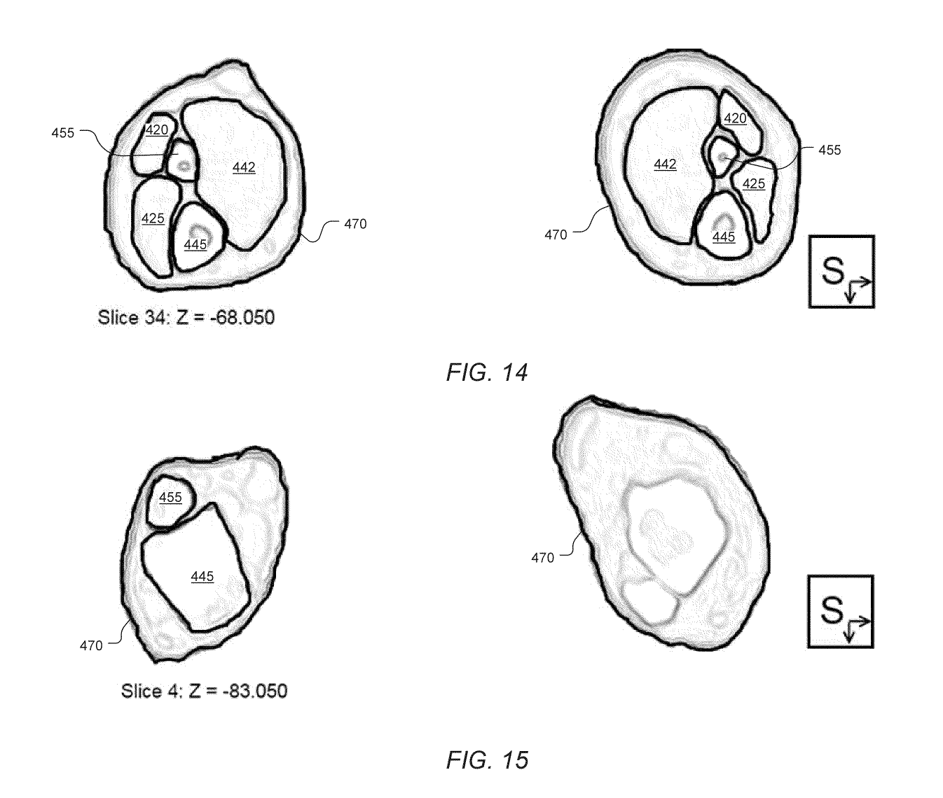

Example images of the three-dimensional (3D) CT atlas of the lower extremity for sarcoma are shown in FIGS. 9 through 15. FIGS. 9 and 10 are an anterior view and a right view, respectively, of a 3D musculoskeletal model of the lower extremity. FIGS. 11 through 15 are CT slices of the lower extremity emphasizing the contours of the relevant musculoskeletal structures.

The invention also includes a method of deforming (transforming and/or morphing) data from the 3D atlas onto a diagnostic image of a. subject of interest. In this way, the practitioner can easily correlate the anatomy of the subject, either in real life or on the diagnostic image, with the 3D atlas.

The methods and apparatuses described herein can improve the performance of interventions by taking advantage of transformations between the anatomy of an individual patient and an atlas. They can be useful in improving any of the four paradigms of intervention. The methods use a rigid and a non-rigid, or deformable, transformation between the atlas and either the anatomy of an individual patient or one or more images of the anatomy of an individual patient, or a combination thereof. This can provide a physician with information otherwise unavailable.

An atlas is defined here, for the purposes of this description, as a computer-readable description of anatomical information. The anatomical information may include images and geometrical entities and annotations and other information. An image may be: a one-dimensional image, such as an ultrasound echo or an X-ray line; a two-dimensional image, such as a plain X-ray image or an ultrasound image or a digitally reconstructed radiograph (DRR) formed from a three-dimensional image; a three-dimensional image, such as a computed tomography scan or a magnetic resonance image or a three-dimensional ultrasound image or a time sequence of two-dimensional images; or a four-dimensional image, such as a time sequence of three-dimensional images; or any other information that may be interpreted as an image. Geometrical entities may be: points; curves; surfaces; volumes; sets of geometrical entities; or any other information that may be interpreted as a geometrical entity. An annotation may be: material properties; physiological properties; radiological absorptiometric properties. An atlas, therefore, is a form of spatial database that can be queried and updated.

A transformation is a mathematical mapping of a point or an object in a first coordinate set C.sub.1 to a point or object in a second coordinate set C.sub.2. A transformation of a point can be represented as y=T(x) where x is a point in C.sub.1 and y is the point in C.sub.2 to which x is transformed. A transformation of every point in a first coordinate set to one or more points in a second coordinate set is a transformation from the first coordinate set to the second coordinate set. A transformation can be continuous or can be discontinuous. An invertible transformation is a transformation of a point in a first coordinate set C.sub.1 to a point in a second coordinate set C.sub.1, represented as y=T(x), such that there exists an inverse transformation x=T.sup.−1(y).

A parameterized transformation is a transformation in which mathematical entities called parameters take specific values; a parameter is a mathematical entity in the transformation other than the point in the first coordinate set that is transformed to a point in a second coordinate set so, for example, in the above definition of a rigid transformation both R and t are parameters of the rigid transformation. A parameter can vary continuously, in which case there are an infinite number of transformations specified by the parameter. A parameter can vary discretely, in which case there is a finite number of transformations specified by the parameter.

In the present invention morphing atlas data from the atlas coordinate set to the patient coordinate set is accomplished by performing a rigid registration between the at least one landmark identified in the patient coordinate set and the corresponding obtained atlas data in the atlas coordinate set and by performing a non-rigid registration of the musculoskeletal structure divided into the relevant contoured fascial compartments, skeletal structures and vascular structures while maintaining the rigid registration of the at least one landmark. Rigid registrations or transformation include rotations, translations, reflections, or their combination. In contrast, non-rigid registrations or transformations involve changes in the shape and/or size of a figure. As such, in accordance with the present invention, one or more landmarks are used to first perform a rigid registration between the atlas and the patient data and then the remaining, interval, musculoskeletal structure is morphed using anon-rigid transformation between the atlas and the patient data.

A morph is either an invertible deformable parameterized transformation or the result of applying an invertible deformable parameterized transformation to a set of points in a first coordinate set that maps to another set of points, whether in the same coordinate set or in a second coordinate set. Whether the term refers to the transformation itself or to its application to a set of points, is understood from the context of usage by a practitioner of the art. In any embodiment the inverse of the deformable parameterized transformation may be found analytically or numerically or by any other means of inverting a transformation.

The method uses anatomical structures as points, or landmarks, for morphing the image from the 3D atlas. Illustrative structures include, but are not limited to, bone structure and soft tissue structure.

The methods and apparatuses described herein use a morph or morphs for the purpose of providing computer-assisted intervention guidance. The methods and apparatuses are applicable to all four of the current paradigms for computer-assisted intervention, each of which will be described. The methods and apparatuses use morphing to establish a correspondence between an atlas and a patient, which is useful because information related to a geometric entity in the atlas can be related to the location of the morphed geometric entity in a patient coordinate set and, because of the invertibility of the morphing transformation, vice versa.

The use of morphing extends the preoperative-image paradigm by providing atlas information to the physician using the system. The atlas information is provided by morphing an atlas to the patient, or to a preoperative image, or to both, for the purpose of intraoperative guidance. The morphing transformation from the atlas to the patient can be calculated using data collected from the patient's anatomical surfaces, or data inferred from the patient's anatomy, or both forms of data, and data from the atlas. The morphing transformation from the atlas to a preoperative image can be calculated using data derived from the preoperative image and data from the atlas. The use of preoperative images in conjunction with the atlas can provide a better morph of the atlas to the patient.

Morphing for guidance using a preoperative image or images of a patient can be explained by way of an example of how knee surgery might be performed. Supposition that an atlas of the human left knee has been developed by merging several detailed scans of volunteer subjects by both computed tomography imaging and magnetic resonance imaging, with annotated information in the atlas provided by a practitioner skilled in the art of interpreting medical images. The annotations could include surface models of the bones, the mechanical center of the distal femur, the mechanical center of the femoral head, the mechanical axis that joins the centers, the transepicondylar axis, the insertion sites of the cruciate and collateral ligaments, the neutral lengths of the ligaments, and numerous other points and vectors and objects that describe clinically relevant features of the human left knee. Prior to surgery a preoperative CT image of the patient's right knee could be acquired by CT scanning. The atlas images of the left knee could be morphed to the preoperative image of the patient's right knee by many means, such as point-based methods that minimize a least-squares disparity function, volumetric methods that maximize mutual information, or any other methods of determining a morphing transformation. The morph would need to include reflection about a plane to morph a left knee to a right knee, an example of such a plane being the sagittal plane.

During a surgical intervention, for example, a physician could determine a plurality of points on the surface of a patient's right femur, the points measured in a patient-based coordinate set. A registration transformation can then be calculated between the preoperative image and the points in a patient coordinate set, such that a disparity function of the points and the surface models is minimized. The morph transformation from an atlas coordinate set to the preoperative image can then be composed with the registration transformation to provide a morph transformation from an atlas coordinate set to a patient coordinate set. Using the morph transformation, a point in an atlas coordinate set can be morphed into a patient coordinate set. The morphed point can be used in many ways, such as to determine the distance of the morphed point from one of the annotated axes, which provides to a physician an estimate of the location of an axis in a patient where the axis might be difficult to estimate directly from the patient. A computer program can then provide to the physician images derived from the preoperative image, and images and annotations derived from the atlas, to improve the physician's ability to plan and perform the surgical procedure.

In an illustrative embodiment for providing interventional guidance with preoperative images of a patient, a computer program communicates with a tracking system and can access one or more preoperative images and an atlas. A preferred embodiment utilizes a configuration having a first tracked device with coordinate set is attached to a patient and a tracking system provides to a computer program in computer the position of the first tracked device. In the preferred embodiment position is in the coordinate set of the first tracked device in an alternative embodiment this position is provided in a second coordinate set. A second tracked device is attached to an actual instrument. In the preferred embodiment the position of the second tracked device with coordinate set is provided to the computer program in coordinate set of the first tracked device. In an alternative embodiment the position of the tracked device is provided to the computer program in the second coordinate set and the computer program computes the relative position of the second tracked device with respect to the coordinate set of the first tracked device.

As a physician directly contacts surfaces of anatomical regions of the patient and the tracking system, or the computer program, or both, can determine the position of the guidance point on the actual instrument in the coordinate set of the first tracked device, so that the coordinate set of the first tracked device acts as the coordinate set of the patient.

A method, additionally embodied in the computer program, is shown that can be used for morphed guidance with an atlas image, in which the morph transformation from the atlas coordinate set to the patient coordinate set and position of the tracked actual instrument from the coordinate set relative to the patient coordinate set can be combined with a morph or registration transformation from a coordinate set of a preoperative image.

A morph transformation and tracking of the actual instrument can be used to morph an atlas image and superimpose an image of a virtual instrument on a morphed slice of the atlas image, in combination or separate from use of a registration transformation and tracking of the actual instrument can be used to show a preoperative image and to superimpose an image of a virtual instrument on a morphed slice of the preoperative image.

In the preferred embodiment of the computer program one or more morph transformations are calculated from the coordinate set or sets of the atlas to the coordinate set or sets of the preoperative image or images. A parameterization of a rigid transformation from the coordinate set of a preoperative image to the coordinate set of the patient is formulated. The parameters of the rigid transformation are calculated on as to minimize a disparity function between the transformed data in the preoperative image and corresponding data in the patient coordinate set. The resulting registration can be mathematically and numerically composed with a morph from an atlas coordinate set to a preoperative-image coordinate set and thus provide a morph from an atlas coordinate set to the patient coordinate set.

Preferred embodiments can include coordinate transformations in which registration transformation from a coordinate set of a preoperative image to coordinate set of the patient is calculated from patient data, and morph transformation from a coordinate set of an atlas to a coordinate set of a preoperative image is calculated from image data, and morph transformation from a coordinate set of an atlas to coordinate set of the patient is composed from the other two transformations, and relative position of the coordinate set of a tracked actual instrument is provided from information provided by a tracking system. By means of these calculations the method provides morphs from an atlas to a patient and morphs from an atlas to a preoperative image, as well as registrations from a preoperative image to a patient.

In a first alternative embodiment for providing interventional guidance with preoperative images of a patient, the surface points in the patient coordinate set are used as data to determine one or more rigid transformations between the coordinate set or sets of the preoperative image or images and the patient coordinate set. The patient data are also used to determine one or more morph transformations from the coordinate set or sets of the atlas to the patient coordinate set.

The coordinate transformations of the first alternative embodiment include registration transformation from a coordinate set of a preoperative image to coordinate set of the patient is calculated from patient data and morph transformation from a coordinate set of an atlas to a coordinate set of a preoperative image is calculated from image data and morph transformation from a coordinate set of an atlas to coordinate set of the patient is calculated from patient data and relative position of the coordinate set of a tracked actual instrument is provided from information provided by a tracking system. By means of these calculations the method provides morphs from an atlas to a patient and morphs from an atlas to a preoperative, as well as registrations from a preoperative image to an atlas.

In a second alternative embodiment for providing interventional guidance with preoperative images of a patient, one or more morph transformations are calculated from the coordinate set or sets of the atlas to the coordinate set or sets of the preoperative image or images. In the second alternative embodiment the surface points in the patient coordinate set are used as data to determine one or more morph transformations from the coordinate set or sets of the atlas to the patient coordinate set.

The coordinate transformations of the second alternative embodiment in which morph transformation from a coordinate set of an atlas to a coordinate set of a preoperative image is calculated from image data and morph transformation from a coordinate set of an atlas to coordinate set of the patient is calculated from patient data and morph transformation from a coordinate set of a preoperative image to coordinate set of the patient is calculated from the other two transformations and relative position of the coordinate set of a tracked actual instrument is provided from information provided by a tracking system. By means of these calculations the method provides morphs from an atlas to a patient and morphs from an atlas to a preoperative image and morphs from a preoperative image to a patient.

In a third alternative embodiment for providing interventional guidance with preoperative images of a patient, the surface points in the patient coordinate set are used to determine one or more rigid transformations between the coordinate set or sets of the preoperative image or images and the patient coordinate set. The surface points data are also used to determine one or more morph transformations from the coordinate set or sets of the atlas to the patient coordinate set. The resulting registration can be mathematically and numerically composed with a morph from an atlas coordinate set to the patient coordinate set and thus provide a morph from an atlas coordinate set to a preoperative-image coordinate set.

The coordinate transformations of the third alternative embodiment include registration transformation from a coordinate set of a preoperative image to coordinate set of the patient is calculated from patient data and morph transformation from a coordinate set of an atlas to coordinate set of the patient is calculated from patient data and morph transformation from a coordinate set of an atlas to a coordinate set of a preoperative image is calculated from the other two transformations and relative position of the coordinate set of a tracked actual instrument is provided from information provided by a tracking system. By means of these calculations the method provides morphs from an atlas to a patient and morphs from an atlas to a preoperative image, as well as registrations from a preoperative image to a patient.

In a fourth alternative embodiment for providing interventional guidance with preoperative images of a patient, the surface points in the patient coordinate set are used as data to determine one or more rigid transformations between the coordinate set or sets of the preoperative image or images and the patient coordinate set. The surface data are also used to determine one or more morph transformations from the coordinate set or sets of the atlas to the patient coordinate set.

The coordinate transformations of the fourth alternative embodiment include registration transformation from a coordinate set of a preoperative image to coordinate set of the patient is calculated from patient data and morph transformation from a coordinate set of an atlas to coordinate set of the patient is calculated from patient data and relative position of the coordinate set of a tracked actual instrument is provided from information provided by a tracking system. By means of these calculations the method provides morphs from an atlas to a patient and registrations from a preoperative image to a patient.

In a fifth alternative embodiment for providing interventional guidance with preoperative images of a patient, one or more morph transformations are calculated from the coordinate set or sets of the alas to the coordinate set or sets coordinate set of the preoperative image or images. In the fifth alternative embodiment the surface points in the patient coordinate set are used as data to determine one or more morph transformations from the coordinate set or sets of the atlas to the patient coordinate set.

The coordinate transformations of the fifth alternative embodiment include morph transformation from a coordinate set of an atlas to a coordinate set of a preoperative image is calculated from image data and morph transformation from a coordinate set of an atlas to coordinate set of the patient is calculated from patient and relative position of the coordinate set of a tracked actual instrument is provided from information provided by a tracking system. By means of these calculations the method provide morphs from an atlas to a patient and morphs from an atlas to a preoperative image.

The computer program, or another computer program, can subsequently relate the location of the tracked actual instrument or of another tracked actual instrument to the atlas. In the preferred embodiment, the computer program morphs images and other atlas data to the coordinate set of the patient, and displays these images and data to the physician with a computer representation of the tracked actual instrument superimposed upon these images and data. By this method the physician can use the images and data to guide a tracked actual instrument within the patient's body. In an alternative embodiment, the computer program morphs the coordinate set of the patient to the coordinate set or sets of the atlas by means of the inverse of the morph transformation from the atlas coordinate set or sets to the patient coordinate set, and displays atlas images and data to the physician with a computer representation of the deformed tracked actual instrument superimposed upon these images and data.

Other data determined in the coordinate set of the patient can be used to morph an atlas to a patient, as described in the use of the preferred embodiment for guidance without images. A morphing transformation can be used to provide atlas data to an interventionalist, as described in the use of the preferred embodiment for guidance without images.

The present invention may be embodied on various computing platforms that perform actions responsive to software-based instructions. The following provides an antecedent basis for the information technology that may be utilized to enable the invention.

The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object oriented programming language such as Java, C#, C++ or the like and conventional procedural programming languages, such as the “C” programming language or similar programming languages.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A method for the treatment of a sarcoma in a patient using radiotherapy, the method, comprising:
providing a computer readable atlas of a musculoskeletal structure having a data set representing a three dimensional model of the musculoskeletal structure divided into the relevant contoured fascial compartments, skeletal structures and vascular structures and comprising the relevant musculoskeletal anatomic terminology;
obtaining atlas data in an atlas coordinate set from the atlas;
obtaining patient data in a patient coordinate set that corresponds to obtained atlas data in the atlas coordinate set, the patient data including the sarcoma;
identifying at least one landmark in the patient coordinate set;
morphing atlas data from the atlas coordinate set to the patient coordinate set by performing a rigid registration between the at least one landmark identified in the patient coordinate set and the corresponding Obtained atlas data in the atlas coordinate set and by performing a non-rigid registration of the musculoskeletal structure divided into the relevant contoured fascial compartments, skeletal structures and vascular structures while maintaining the rigid registration of the at least one landmark; and
providing an image of the patient coordinate set with the morphed atlas data to an image-guided radiation therapy device for the treatment of the sarcoma using radiotherapy.

2. The method of claim 1, wherein the step of obtaining patient data in a patient coordinate set that corresponds to atlas data in the atlas coordinate set comprises the step of collecting a plurality of points in a patient coordinate set from the patient that correspond to points in an atlas coordinate set from the atlas.

3. The method of claim 1, wherein the obtained patient data comprises a plurality of points from the patient anatomy in a patient coordinate set, and the obtained atlas data comprises a plurality of points from the atlas in an atlas coordinate set.

4. The method of claim 3, wherein the step of obtaining a plurality of points in a patient coordinate set that correspond to points in an atlas coordinate set from the atlas comprises:
obtaining an image of the patient including a plurality of points in an image coordinate set that correspond to points in an atlas coordinate set from the atlas;
collecting a plurality of points in a patient coordinate set from the patient that correspond to points in an atlas coordinate set from the atlas; and
collecting a plurality of points in a patient coordinate set from the patient that correspond to points in an image coordinate set from the image.

5. The method of claim 1, wherein the plurality of points correspond to bone structure.

6. The method of claim 3, wherein the plurality of points correspond to soft tissue structure.

7. The method of claim 1, wherein the at least one landmark corresponds to a bone structure.

8. The method of claim 1, wherein the at least one landmark corresponds to a soft tissue structure.

9. The method of claim 1, wherein the data set of the computer readable atlas further comprises the radiological absorptiometric properties of the musculoskeletal anatomy.

10. One or more non-transitory computer-readable media having computer-executable instructions for performing a method of running a software program on a computing device for the treatment of a sarcoma in a patient using radiotherapy, the computing device operating under an operating system, the method including issuing instructions from the software program comprising:
providing a computer readable atlas of a musculoskeletal structure having a data set representing a three dimensional model of the musculoskeletal structure divided into the relevant contoured fascial compartments, skeletal structures and vascular structures and comprising the relevant musculoskeletal anatomic terminology;
obtaining atlas data in an atlas coordinate set from the atlas;
obtaining patient data in a patient coordinate set that corresponds to obtained atlas data in the atlas coordinate set, the patient data including the sarcoma;
identifying at least one landmark in the patient coordinate set;
morphing atlas data from the atlas coordinate set to the patient coordinate set by performing a rigid registration between the at least one landmark identified in the patient coordinate set and the corresponding obtained atlas data in the atlas coordinate set and by performing a non-rigid registration of the musculoskeletal structure divided into the relevant contoured fascial compartments, skeletal structures and vascular structures while maintaining the rigid registration of the at least one landmark; and
providing an image of the patient coordinate set with the morphed atlas data to an image-guided radiation therapy device for the treatment of the sarcoma using radiotherapy.

11. The media of claim 10, wherein the step of obtaining patient data in a patient coordinate set that corresponds to atlas data in the atlas coordinate set comprises the step of collecting a plurality of points in a patient coordinate set from the patient that correspond to points in an atlas coordinate set from the atlas.

12. The media of claim 10, wherein the obtained patient data comprises a plurality of points from the patient anatomy in a patient coordinate set, and the obtained atlas data comprises a plurality of points from the atlas in an atlas coordinate set.

13. The media of claim 12, wherein the step of obtaining a plurality of points in a patient coordinate set that correspond to points in an atlas coordinate set from the atlas comprises:
obtaining an image of the patient including a plurality of points in an image coordinate set that correspond to points in an atlas coordinate set from the atlas;
collecting a plurality of points in a patient coordinate set from the patient that correspond to points in an atlas coordinate set from the atlas; and
collecting a plurality of points in a patient coordinate set from the patient that correspond to points in an image coordinate set from the image.

14. The media of claim 10, wherein the plurality of points correspond to bone structure.

15. The media of claim 10, wherein the plurality of points correspond to soft tissue structure.

16. The media of claim 10, wherein the at least one landmark corresponds to a bone structure.

17. The media of claim 10, wherein the at least one landmark corresponds to a soft tissue structure.

18. The media of claim 10, wherein the data set of the computer readable atlas further comprises the radiological absorptiometric properties of the musculoskeletal anatomy.
